SEQUENCE LISTING (3 sequences)

SEQ ID NO: 1 - DNA, 19 bp, synthetic construct
  source 1..19: mol_type = other DNA
ggtccgtgtttcaagacgg

SEQ ID NO: 2 - DNA, 588 bp, synthetic construct
  source 1..588: mol_type = other DNA
tttacggcattccctagtagcggcgagcgaagcgggaagagctcaaatttataatctggcaccttcggtgtccgagttgtaatctctagaaatgttttccgcgctggaccgcacacaagtctgttggaatacagcggcatagtggtgagacccccgtatatggtgcggacgcccagcgctttgtgatacattttcgaagagtcgagttgtttgggaatgcagctcaaattgggtggtaaattccatctaaagctaaatattggcgagagaccgatagcgaacaagtaccgtgagggaaagatgaaaagcactttggaaagagagttaacagtacgtgaaattgttggaagggaaacgcttgaagtcagacttgcttgccgagcaatcggtttgcaggccagcatcagttttccgggatggataatggtagagagaaggtagcagtttcggctgtgttatagctctctgctggatacatcttgggggactgaggaacgcagtgtgcctttggcgggggtttcgacctcttcacacttaggatgctggtggaatggctttaaacgacccgtcttgaaacacggacccaaa

SEQ ID NO: 3 - DNA, 24 bp, synthetic construct
  source 1..24: mol_type = other DNA
gcatatcaataagcggaggaaaag

(19) United States
(12) Patent Application Publication
(10) Pub. No.: US 2024/0225056 A1
(43) Pub. Date: Jul. 11, 2024

(54) Rhodotorula Capable of Efficiently Degrading Ethyl Carbamate and Application Thereof

(71) Applicant: Dalian Polytechnic University, Dalian (CN)

(72) Inventors: Sufang ZHANG, Dalian (CN); Siyu XUE, Dalian (CN); Beiwei ZHU, Dalian (CN); Yingxi CHEN, Dalian (CN); Huipeng LIANG, Dalian (CN); Xinping LIN, Dalian (CN); Chaofan JI, Dalian (CN); Liang DONG, Dalian (CN); Yiwei DAI, Dalian (CN)

(21) Appl. No.: 18/589,595

(22) Filed: Feb. 28, 2024

Related U.S. Application Data
(63) Continuation of application No. PCT/CN2023/102849, filed on Jun. 27, 2023.

(30) Foreign Application Priority Data
Aug. 16, 2022 (CN) 2022109858864

(51) Int. Cl. A23L 5/20; A23C 9/127; A23L 23/00; A23L 27/50; C12H 1/12; C12J 1/00; C12N 1/16; C12R 1/645

(52) U.S. Cl. CPC A23L 5/28; A23C 9/127; A23L 23/00; A23L 27/50; C12H 1/12; C12J 1/00; C12N 1/165; C12R 2001/645

(57) ABSTRACT

Disclosed are Rhodotorula capable of efficiently degrading ethyl carbamate and application thereof, belonging to the technical field of food biotechnology. The present disclosure provides Rhodotorula DL-XSY01 capable of efficiently degrading ethyl carbamate (EC), with a preservation number being CGMCC No. 23534. The Rhodotorula DL-XSY01 is screened, identified, activated, fermented, and embedded to obtain an EC degradation preparation. The strain DL-XSY01 obtained by the present disclosure is prepared into a degradation agent after embedding, which can be used for removing EC in fermented foods. Furthermore, the present disclosure utilizes the degradation agent derived from strain DL-XSY01 to eliminate EC from various food systems. It demonstrates significant EC removal efficacy, low production costs, ease of use, and easy removal from food systems. Thus, it finds wide applicability in fermented foods such as alcoholic beverages, fermented dairy products, soy sauce, and vinegar.

### REFERENCE TO SEQUENCE LISTING

The instant application contains a Sequence Listing in XML format as a file named “YGHY-2023-23-SEQ.xml”, created on Feb. 6, 2024, of 5 kB in size, and which is hereby incorporated by reference in its entirety.

### TECHNICAL FIELD

The present disclosure relates to Rhodotorula capable of efficiently degrading ethyl carbamate and application thereof, belonging to the technical field of food biotechnology.

### BACKGROUND

Ethyl carbamate (EC) is a genotoxic and highly carcinogenic metabolite widely found in alcoholic beverages and fermented foods (such as fermented bean curd, soy sauce, cheese, vinegar, and pickles).

In recent years, scholars from all over the world have discovered the presence of EC in distilled liquor, Huangjiu (Chinese rice wine) and wine, among which the EC content in the distilled liquor and Huangjiu is relatively high, while the EC content in the wine is lower. EC has antibacterial and anti-tumor activities. In the early 1940s, EC was used as an anesthetic drug; in 1943, some scholars found that EC was carcinogenic, but it did not attract enough attention; and in 1974, the International Agency for Research on Cancer listed EC as a Class 2B carcinogen, and upgraded same to a Class 2A carcinogen in 2007. Meanwhile, EC has been proven to have multi-site carcinogenicity, which can lead to lung cancer, lymphatic cancer, liver cancer, skin cancer, and the like. In experiments with animal models, transcription factors STAT3, NF-kB, and extracellular signaling protein kinase (ERK) have been shown to be involved in the development of EC-induced tumors. On the other hand, EC has also been shown to induce the death of human liver cancer cells HepG2 by inducing oxidative stress response, reducing detoxification ability, consuming energy, disrupting membrane structural integrity, damaging DNA and proteins, and the like. At present, according to the data from the FAO/WHO Joint Committee on Food Additives, the average amount of EC ingested by the human body through brewing wine is 65 ng·kg⁻¹·d⁻¹, which is five times higher than that of other fermented foods.

EC is mainly produced by reacting urea, citrulline, carbamoyl phosphate, and other precursors with ethanol. The control methods of EC mainly include the limitation of precursors and the reduction of EC. Urease digestion of urea is mostly used for the limitation of precursors, but urease specificity is single, so that precursor types and applicability of different systems need to be considered, and practicability is limited. However, the reduction of EC has a wide range of applications and thus is more commonly used. This method generally implements the process of hydrolyzing EC into ethanol, ammonia and carbon dioxide through the action of EC hydrolase and enzyme-producing microorganisms.

Tian Yaping et al. screened and isolated an EC degrading strain Penicillium variabile from the mouse gastrointestinal tract, isolated EC hydrolase from the strain, and found that the EC hydrolase can effectively degrade EC in Baijiu (Chinese liquor).

As for the strains separated from a Baijiu brewing process, reported are: Lysinibacillus sphaericus MT3 isolated from a Baijiu fermentation process by Xu Yan et al., and Rhodotorula mucilaginosa isolated from a Chinese Baijiu brewing process by Wu Qun et al. The resource library of EC degrading strains derived from fermented food needs to be expanded urgently.

Therefore, the present disclosure performs screening, separation, identification and application characterization on microorganisms capable of degrading EC in fermented foods, which can expand the resource library of EC degrading strains derived from fermented foods, provide effective means for reducing EC in fermented foods, and provide technical support to ensure the safety of fermented food.

### SUMMARY

The purpose of the present disclosure is to provide a yeast strain DL-XSY01 isolated from fermented food, and a degradation agent prepared by using same.

Rhodosporidium toruloides DL-XSY01 has been deposited in the China General Microbiological Culture Collection Center (CGMCC) on Oct. 8, 2021, with preservation number CGMCC No. 23534.

The R. toruloides DL-XSY01 was isolated and collected from the vinegar fermented grains of Shandong Xiaomi Vinegar Factory, and the sequencing results were subjected to Blastn analysis. It was found that the strain had the highest homology with R. toruloides and R. mucilaginosa, both reaching 99%. According to morphology and 26s rRNA identification, the strain DL-XSY01 was identified as R. toruloides and named R. toruloides DL-XSY01.

The R. toruloides DL-XSY01 grew well on a YPD solid plate, and formed moist, round, protruding, smooth, and light pink colonies after being cultured at 28° C. for 48 h. Microscopic examination revealed that the strain was elliptical and long rod-shaped, which showed orange red after standing at 4° C. for 2 d. The optimum growth temperature was 28° C., and the optimum pH was 5-6.

The present disclosure provides application of a strain of R. toruloides DL-XSY01 or its fermentation broth in the preparation of a product capable of degrading ethyl carbamate.

In one embodiment of the present disclosure, the product is either a chemical or a microbial agent.

In one embodiment of the present disclosure, a preparation method of the microbial agent includes:
(1) inoculating the R. toruloides DL-XSY01 into a YPD liquid medium for activation, and carrying out constant temperature shake cultivation at 25-32° C. for 24-48 h at a speed of 150-250 rpm to obtain seed fermentation broth;
(2) inoculating the seed fermentation broth into a fermentation medium at an inoculation amount of 10%-20%, and fermenting until the total bacterial count is 3×10⁸-1.8×10⁹ cfu/mL under the conditions that the temperature is 25-32° C., the ventilation rate is 0.6-1.0 m³/min and the stirring rate is 150-250 rpm, to obtain a fermentation product of the R. toruloides DL-XSY01;
(3) embedding the fermentation product of the R. toruloides DL-XSY01 prepared in step (2) to obtain the microbial agent.

In one embodiment of the present disclosure, the addition amount of the R. toruloides DL-XSY01 in the product is at least 3×10⁸ cfu/mL.

In one embodiment of the present disclosure, the chemical includes, but is not limited to, an ethyl carbamate degradation agent, an ethyl carbamate adsorbent, an ethyl carbamate inhibitor, an ethyl carbamate decomposing agent, an ethyl carbamate degrading microbial strain, or an ethyl carbamate degrading microbial agent.

The present disclosure further provides a method for degrading ethyl carbamate in food. During preparation of the food, the R. toruloides DL-XSY01 or its fermentation broth is added to degrade ethyl carbamate, and the degraded product is sterilized to obtain the food.

In one embodiment of the present disclosure, the food includes, but is not limited to, a fermented food or an alcoholic beverage.

In one embodiment of the present disclosure, the food is Baijiu, Huangjiu, red wine, yogurt, vinegar, or soy sauce.

The present disclosure further provides an ethyl carbamate degradation agent. The degradation agent is prepared according to the following method:
(1) inoculating the R. toruloides DL-XSY01 into a YPD liquid medium for activation, and carrying out constant temperature shake cultivation at 25-32° C. for 24-48 h at a speed of 150-250 rpm to obtain seed fermentation broth;
(2) inoculating the seed fermentation broth into a fermentation medium at an inoculation amount of 10%-20%, and fermenting until the total bacterial count is 3×10⁸-1.8×10⁹ cfu/mL under the conditions that the temperature is 25-32° C., the ventilation rate is 0.6-1.0 m³/min and the stirring rate is 150-250 rpm, so as to obtain a fermentation product of the R. toruloides DL-XSY01;
(3) embedding the fermentation product of the R. toruloides DL-XSY01 prepared in step (2) to obtain the ethyl carbamate degradation agent.

In one embodiment of the present disclosure, the fermentation medium is a YPD liquid medium (prepared by adding 20.0 g of peptone, 10.0 g of yeast powder, and 20.0 g of glucose, filling distilled water up to 1 L, adjusting pH to 7.0, and carrying out high-pressure sterilization for 20 min).

In one embodiment of the present disclosure, the embedding method includes:
S1. mixing sodium alginate (4%) with bacteria-containing fermentation broth, cell lysate and bacterial cell suspension at a ratio of 1:1;
S2. using a 10 ml syringe to aspirate and slowly drip into a curing solution (a saturated boric acid solution containing 0.6% CaCl₂)) at a rate of 2-10 drops per second, with a curing time being about 5 h;
S3. rinsing the immobilized cells for 3-4 times with normal saline, and placing same in a chitosan (2%) solution for 40 min of film coating;
S4. rinsing again with 0.8% normal saline for 3-4 times, draining off the water, and storing at 4° C. for later use.

In one embodiment of the present disclosure, the particle sizes of microspheres in a wet state are 3-5 mm.

In one embodiment of the present disclosure, the fermentation product of the R. toruloides DL-XSY01 described in step (2) includes: bacteria-containing fermentation broth of R. toruloides DL-XSY01, fermentation broth supernatant of R. toruloides DL-XSY01, bacterial cell suspension of R. toruloides DL-XSY01, or cell lysate of R. toruloides DL-XSY01.

In one embodiment of the present disclosure, the bacteria-containing fermentation broth of R. toruloides DL-XSY01 is prepared by drying the fermentation product of the R. toruloides DL-XSY01, and blending the product into 1/20 of the original volume with deionized water;
the bacterial cell suspension of R. toruloides DL-XSY01 is obtained by centrifuging the fermentation product of the R. toruloides DL-XSY01 at a speed of 4000 g for 10 min and collecting bacterial cell sediment, and diluting until the concentration is 200D₆₀₀;
the cell lysate of R. toruloides DL-XSY01 is obtained by breaking the cell wall of the bacterial cell suspension of R. toruloides DL-XSY01.

In one embodiment of the present disclosure, the drying method includes freeze-vacuum drying.

In one embodiment of the present disclosure, the method of preparing immobilized microspheres after embedding as described in step (3) includes the following steps:
S1. mixing sodium alginate (4%) with bacteria-containing fermentation broth, cell lysate and bacterial cell suspension at a ratio of 1:1;
S2. using a 10 ml syringe to aspirate and slowly drip the mixture into a curing solution (a saturated boric acid solution containing 0.6% CaCl₂)) at a rate of 2-10 drops per second, with a curing time being about 5 h;
S3. rinsing the immobilized cells for 3-4 times with normal saline, and placing them in a chitosan (2%) solution for 40 min of film coating;
S4. rinsing again with 0.8% normal saline for 3-4 times, draining off the water, and storing at 4° C. for later use.

In one embodiment of the present disclosure, the particle sizes of microspheres in a wet state are 3-5 mm.

The present disclosure also provides application of the R. toruloides DL-XSY01 in the degradation of ethyl carbamate.

### Beneficial Effects

(1) The present disclosure obtains a strain DL-XSY01 capable of efficiently degrading EC from millet vinegar fermented grains, which can grow using EC as the sole carbon source and effectively degrade EC. The degradation agent exhibits an EC degrading rate reaching up to 76%, thus having a good EC biodegradation effect.

(2) Since the strain DL-XSY01 obtained by the present disclosure is separated from the fermented food, the strain and the EC degradation agent prepared therefrom can be used to remove EC from the fermented food, and the EC degrading rate in Baijiu can reach 76%. The method provided by the present disclosure has the characteristics of low production cost, convenient use, and easy removal from food systems, and can effectively improve the quality and safety of fermented foods such as alcoholic drinks, yogurt, soy sauce, and vinegar.

(3) The safety evaluation indicates that the strain DL-XSY01 is safe. In terms of antibiotic sensitivity, the strain is moderately sensitive to the antibiotic cephalexin, and is sensitive to 10 antibiotics: cefazolin, erythromycin, gentamicin, tetracycline, amikacin, ampicillin, streptomycin, vancomycin, minocycline, and penicillin G. In a hemolysis experiment, DL-XSY01 did not show hemolysis (γ-hemolysis) when being cultured on a sheep blood plate at 28° C. However, Staphylococcus aureus ATCC 25923 showed β-hemolysis, and thus a positive control was established. In addition, Escherichia coli Nissle 1917 showed α-hemolysis. Therefore, DL-XSY01 is considered a safe organism that poses no harm to human health.

(4) The probiotic evaluation shows that the strain DL-XSY01 has a good survival rate in artificial simulated gastric juice and artificial simulated intestinal juice; and it has good scavenging ability for DPPH and ABTS free radicals, with scavenging rates researching 90.02% and 93.67%, respectively.

### Biological Material Preservation

A strain of R. toruloides DL-XSY01, classified as R. toruloides, has been deposited in the China General Microbiological Culture Collection Center (CGMCC) on Oct. 8, 2021, with a preservation number being CGMCC No. 23534, and the preservation address is Institute of Microbiology, Chinese Academy of Sciences, No. 3, Yard No. 1, Beichen West Road, Chaoyang District, Beijing.

### BRIEF DESCRIPTION OF FIGURES

FIG. 1 is a photomicrograph of R. toruloides DL-XSY01 according to the present disclosure; and

FIG. 2 is a screening picture of R. toruloides DL-XSY01 according to the present disclosure when EC is used as a carbon source.

### DETAILED DESCRIPTION

The following examples are only preferred specific embodiments of the present disclosures, but the protection scope of the present disclosure is not limited thereto. The present disclosure mainly sets forth strains and application ideas based on the strains. The replacement of simple parameters in the embodiments cannot be described repeatedly one by one in the embodiments, any other changes, modifications, substitutions, combinations, and simplifications that do not deviate from the spiritual essence and principles of the present disclosure should be regarded as equivalent replacement methods, and that made by anyone skilled in the art within the technical scope disclosed in the present disclosure shall be included within the protection scope of the present disclosure.

The present disclosure will be further described below in conjunction with the accompanying drawings and specific examples. Unless otherwise specified, the reagents, methods and equipment used in the present disclosure are conventional reagents, methods and equipment in the art; and unless otherwise specified, the reagents and materials used in the following examples are all commercially available.

Various culture mediums used in the present disclosure are all prepared by conventional methods. If specific experimental conditions and methods are not specified in the molecular biology operations involved in the examples, they shall refer to SambrookJ et al., Science Press, 2002, Guidelines for Molecular Cloning: A Laboratory Manual (Third Edition), or refer to the product manual.

### The Preparation of the Culture Mediums Involved in the Following Examples was as Follows:

YPD liquid medium: prepared by adding 20.0 g of peptone, 10.0 g of yeast powder, and 20.0 g of glucose, filling distilled water up to 1 L, adjusting pH to 7.0, and carrying out high-pressure sterilization for 20 min.

YPD solid medium: prepared by adding 20.0 g of peptone, 10.0 g of yeast powder, 20.0 g of glucose, and 15 g of agar, filling distilled water up to 1 L, adjusting pH to 7.0, carrying out high-pressure sterilization for 20 min, and then pouring onto a plate.

YNB liquid medium: prepared by adding 5000 mg of ammonium sulfate, 2 mg of inositol, 0.4 mg of niacin (vitamin B3), 0.4 mg of thiamine hydrochloride (vitamin B1), 0.04 mg of copper sulfate, 1000 mg of potassium dihydrogen phosphate, 0.5 mg of boric acid, 0.4 mg of pyridoxine hydrochloride (vitamin B6), 0.4 mg of calcium pantothenate (vitamin B5), 0.2 mg of para aminobenzoic acid, 500 mg of magnesium sulfate, 0.4 mg of manganese sulfate, 0.4 mg of zinc sulfate, 0.2 mg of ferric chloride, 0.2 mg of riboflavin (vitamin B2), 100 mg of calcium chloride, 0.1 mg of potassium iodide, 0.2 mg of sodium molybdate, 0.002 mg of biotin (vitamin B7, H), 0.002 mg of folic acid (vitamin B9), and 100 mg of sodium chloride, filling distilled water up to 1 L, and sterilizing with a filter head.

YNB solid medium: prepared by adding 5000 mg of ammonium sulfate, 2 mg of inositol, 0.4 mg of niacin (vitamin B3), 0.4 mg of thiamine hydrochloride (vitamin B1), 0.04 mg of copper sulfate, 1000 mg of potassium dihydrogen phosphate, 0.5 mg of boric acid, 0.4 mg of pyridoxine hydrochloride (vitamin B6), 0.4 mg of calcium pantothenate (vitamin B5), 0.2 mg of para aminobenzoic acid, 500 mg of magnesium sulfate, 0.4 mg of manganese sulfate, 0.4 mg of zinc sulfate, 0.2 mg of ferric chloride, 0.2 mg of riboflavin (vitamin B2), 100 mg of calcium chloride, 0.1 mg of potassium iodide, 0.2 mg of sodium molybdate, 0.002 mg of biotin (vitamin B7, H), 0.002 mg of folic acid (vitamin B9), 100 mg of sodium chloride, and 15 g of agar, filling distilled water up to 1 L, and sterilizing with a filter head.

### The EC Content Assay Method Involved in the Following Examples was as Follows:

The residual amount of EC in culture supernatant was determined using the national standard method GB5009.223-2014, and an inoculation-free YPD medium was set as a control, with 3 replicates for each treatment. The method included the following specific steps: 2 mL of a sample was loaded onto a special column for CleanertEC ethyl carbamate, left to stand for 10 min, washed with 10 ml of n-hexane, dried by a vacuum pump, and eluted with 10 mL of a 5% ethyl acetate-ether solution at a flow rate of 1 mL/min; and the eluent was collected. The received eluent was blown to 0.5 mL with nitrogen at room temperature, and then diluted to 1 mL with methanol. 0.8 g of anhydrous sodium sulfate (100° C., 24 h) was added to the obtained diluent, the product was centrifuged at 10000 rpm, and the supernatant was filtered using a 0.22 μm organic filter membrane. The effluent was available in gas chromatography-mass spectrometry (GC-MS) assay (approximately 0.5 mL).

The GC-MS Assay Conditions were as Follows:

Capillary chromatography column: DB-INNOWAX, 30 m×0.25 mm (inner diameter)×0.25 μm (film thickness); injection port temperature: 220° C.; column temperature: initial temperature 50° C., maintained for 1 min, then increased to 180° C. at a rate of 8ºC/min; after the program operation was completed, running at 240° C. for 5 min; carrier gas: helium gas, purity≥99.999%, with a flow rate being 1.0 mL/min; ion source: El; ionization energy: 70 eV; ion source temperature: 230° C.; Quadrupole temperature: 150° C.; transmission line temperature: 250° C.; solvent delay: 11 min; injection method: injection without stream splitting; injection volume: 1 μL; detection method: selected ion monitor (SIM); and EC selection monitor ion (m/z): 44, 62, 74, 89, quantitative ion 62.

The concentration of ethyl carbamate can be calculated based on a peak area and a standard curve.

Degrading rate calculation formula:

$$Degrading rate = \frac{EC content in control group - EC content in experimental group}{EC content in control group} \times 100 \% .$$

### Example 1: Separation and Preparation of R. toruloides DL-XSY01

### 1. Separation and Purification of Strain:

(1) Sample: Vinegar fermented grains collected from Shandong Xiaomi Vinegar Factory.
(2) The separation and screening method employed antioxidant enrichment first, followed by EC degradation function verification:
1 g of vinegar fermented grains were taken and added into 10 ml of a YPD liquid medium containing 2 mM hydrogen peroxide, the above sample was transferred into a homogenization bag and beat for 30 min, and part of the liquid in the homogeneous bag was pipetted into a 50 mL centrifuge tube under sterile conditions and cultured at 30° C. for 48 h at a speed of 200 rpm. Because the growth of bacteria would be inhibited or even stopped in a strong oxidative environment, and ergothioneine had strong antioxidant properties, strains that could produce higher levels of ergothioneine would grow normally in the medium, while strains that could not produce ergothioneine or had lower yields would be screened out under pressure. After the medium became turbid, diluent was spread on a YPD solid medium. After the liquid was absorbed, the medium was subjected to cultivation upside down at 30° C. for 48 h. Single colonies were selected to be cultured on the YPD solid medium by spread plate method. Contentious purification was performed for three generations, and a single colony was picked out for microscopy. After confirming that there was no contamination, the strain was frozen and preserved, which was named DL-XSY01.

S2 (EC degradation function verification): The strain DL-XSY01 was added to 10 ml of a YNB liquid medium containing 5 g/L EC, cultured at 30° C. and 200 rpm for 24 h, centrifuged at 500 rpm for 5 min, diluted with yeast precipitate, and evenly spread on a YNB solid plate containing 10 g/L EC as the sole carbon source. The YNB solid plate was subjected to cultivation upside down at 28° C. for 72 h to form colonies. It was found that the strain DL-XSY01 could grow well with EC as the sole carbon source, indicating that the strain DL-XSY01 had an EC degradation function.

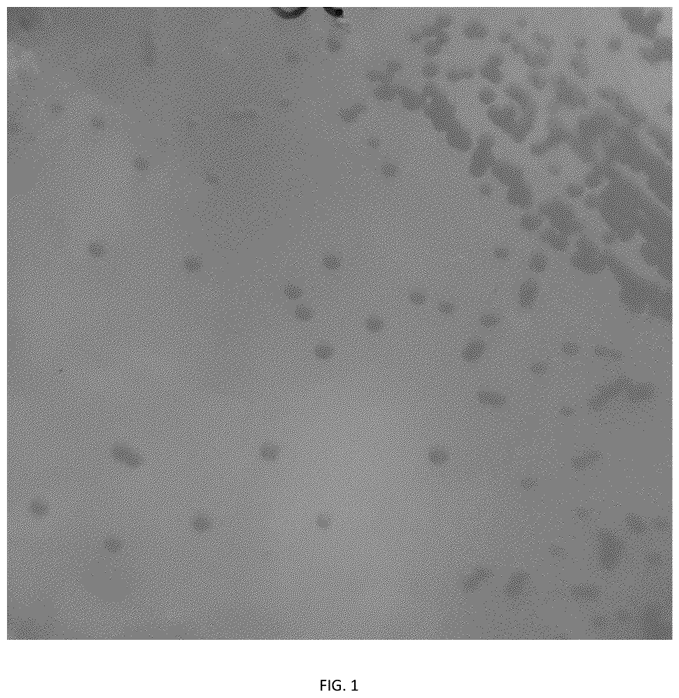

As shown in FIG. 1, it is a growth picture of R. toruloides DL-XSY01 according to the present disclosure when EC is used as a sole carbon source.

### 2. Identification of Strain DL-XSY01:

(1) The genome of strain DL-XSY01 was extracted for 26s rRNA PCR identification, and the genome extraction method was performed for extraction according to a glass bead method in the “Short Protocols in Molecular Biology”.

The PCR Conditions were as Follows:

An amplification system included: 25 μL of 2×Taq Master Mix, 2 μL of a primer D1, 2 μL of a primer D2, 19 μL of sterilized water, and 2 μl of a template. The PCR reaction conditions were: pre-denaturation at 95° C. for 3 min; denaturation at 95° C. for 15 s; annealing at 50° C. for 15 s; extending at 72° C. for 1 min; extending at 72° C. for 15 min; 30 cycles; and circulating indefinitely at 4° C.

After PCR was completed, agarose gel (1.0%) electrophoresis was performed to detect the PCR products in yeast samples. The genomes with bright bands ran out were successfully amplified by PCR, and the successful amplified genomes could be sent for sequencing. Primer sequence D1: GCATATCAATAAGCGGAGGAAAAG (SEQ ID NO.3), and primer sequence D2:

(SEQ ID NO. 1)
GGTCCGTGTTTCAAGACGG.

The 26s rRNA sequence of the strain DL-XSY01 is as follows:

(SEQ ID NO. 2)
TTTACGGCATTCCCTAGTAGCGGCGAGCGAAGCGGGAAGAGCTCAAATT
TATAATCTGGCACCTTCGGTGTCCGAGTTGTAATCTCTAGAAATGTTTT
CCGCGCTGGACCGCACACAAGTCTGTTGGAATACAGCGGCATAGTGGTG
AGACCCCCGTATATGGTGCGGACGCCCAGCGCTTTGTGATACATTTTCG
AAGAGTCGAGTTGTTTGGGAATGCAGCTCAAATTGGGTGGTAAATTCCA
TCTAAAGCTAAATATTGGCGAGAGACCGATAGCGAACAAGTACCGTGAG
GGAAAGATGAAAAGCACTTTGGAAAGAGAGTTAACAGTACGTGAAATTG
TTGGAAGGGAAACGCTTGAAGTCAGACTTGCTTGCCGAGCAATCGGTTT
GCAGGCCAGCATCAGTTTTCCGGGATGGATAATGGTAGAGAGAAGGTAG
CAGTTTCGGCTGTGTTATAGCTCTCTGCTGGATACATCTTGGGGGACTG
AGGAACGCAGTGTGCCTTTGGCGGGGGTTTCGACCTCTTCACACTTAGG
ATGCTGGTGGAATGGCTTTAAACGACCCGTCTTGAAACACGGACCCAAA.

The sequencing results were subjected to Blastn analysis. It was found that the strain had the highest homology with R. toruloides and R. mucilaginosa, both reaching 99%. According to morphology and 26s rRNA identification, the strain DL-XSY01 was R. toruloides, named as R. toruloides DL-XSY01.

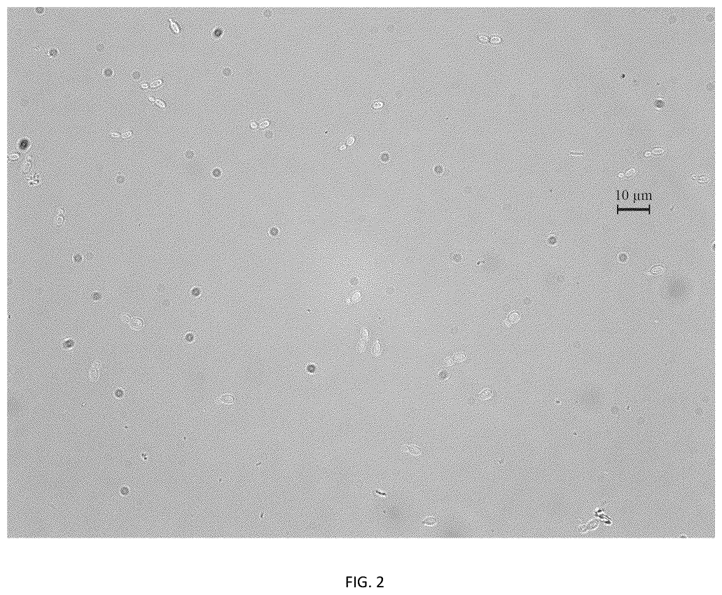

(2) The R. toruloides DL-XSY01 grew well on a YPD solid plate, and formed moist, round, protruding, smooth, and light pink colonies (as shown in FIG. 2) after being cultured at 28° C. for 24-48 h. Microscopic examination revealed that the strain was elliptical and long rod-shaped, which showed orange red after standing at 4° C. for 2 d. The optimum growth temperature was 28° C., and the optimum pH was 5-6.

The EC degrading strain DL-XSY01 was tentatively identified as R. toruloides by morphology and 26s rRNA identification. The strain has been deposited in the China General Microbiological Culture Collection Center (CGMCC) on Oct. 8, 2021, with a preservation number being CGMCC No. 23534. The preservation address is Institute of Microbiology, Chinese Academy of Sciences, No. 3, Yard No. 1, Beichen West Road, Chaoyang District, Beijing.

### Example 2: Preparation of EC Degradation Preparation of Strain R. toruloides DL-XSY01

The Specific Steps were as Follows:
(1) a strain R. toruloides DL-XSY01 was inoculated into a YPD liquid medium for activation, and constant temperature shake cultivation was carried out at 25-30° C. for 24 h at a speed of 150-250 rpm to obtain seed fermentation broth;
(2) the seed fermentation broth was inoculated into a production fermentation tank (70% liquid filling capacity) containing a YPD fermentation medium at an inoculation amount of 10%, with a ventilation rate being 0.6-1.0 m³/min, and a stirring rate being 300 rpm, the cultivation temperature was controlled at 28° C., and cultivation lasted for 48-96 h; after fermentation was finished, a DL-XSY01 culture solution with a total bacterial count of 3×10⁸-1.8×10⁹ cfu/mL was obtained;
after the fermentation was finished, the fermentation broth was collected under sterile conditions, and directly packed into liquid dosage forms with packaging bottles;
(3) after the fermentation was completed, the DL-XSY01 culture solution collected in step (2) was subjected to freeze vacuum drying, and the product was blended into 1/20 of the original volume with deionized water to obtain DL-XSY01 fermentation broth;
(4) after the fermentation was completed, the DL-XSY01 culture solution collected in step (2) was centrifuged at a speed of 4000 g for 10 min to collect DL-XSY01 bacterial cell sediment;
(5) the bacterial cell sediment obtained in step (4) was diluted with a diluent PBS buffer solution, so as to obtain strain DL-XSY01 bacterial cell suspension, where the bacterial concentration was OD₆₀₀=20;
(6) the strain DL-XSY01 bacterial cell suspension obtained in step (5) was subjected to cell wall breaking to obtain DL-XSY01 cell lysate;
(7) the DL-XSY01 bacteria-containing fermentation broth prepared in step (3), the DL-XSY01 bacterial cell suspension prepared in step (5) and the DL-XSY01 cell lysate prepared in step (6) were respectively embedded in sodium alginate to prepare corresponding immobilized microspheres; the specific steps were as follows:
sodium alginate (4%) was mixed with the DL-XSY01 bacteria-containing fermentation broth prepared in step (3), the DL-XSY01 bacterial cell suspension prepared in step (5) and the DL-XSY01 cell lysate prepared in step (6) at a volume ratio of 1:1, respectively; each of the obtained mixtures was sucked with a 10 ml syringe and slowly dripped into a curing solution (a saturated boric acid solution containing 0.6% CaCl₂)) at a rate of 2-10 drops per second, with a curing time being about 5 h, so as to obtain immobilized cells;
the immobilized cells were rinsed for 3-4 times with normal saline, and then placed in a chitosan (2%) solution for 40 min of film coating; each of the products was rinsed again with 0.8% normal saline for 3-4 times, the water was drained off, and the obtained product was stored at 4° C. for later use, so as to obtain EC degradation agents;
embedding preparations prepared from the DL-XSY01 bacteria-containing fermentation broth prepared in step (3), the DL-XSY01 bacterial cell suspension prepared in step (5) and the DL-XSY01 cell lysate prepared in step (6) were respectively the EC degradation agent 1, the EC degradation agent 2, and the EC degradation agent 3;
(8) the bacterial concentrations of the DL-XSY01 in the EC degradation agent 1, the EC degradation agent 2, and the EC degradation agent 3 were respectively detected, which were OD₆₀₀=10, OD₆₀₀=20 and OD₆₀₀=0, respectively.

### Example 3: Application of EC Degradation Agent Prepared from R. toruloides DL-XSY01 in the Degradation of EC in Baijiu

The Specific Steps were as Follows:
each of 20 ml of the EC degradation agent 1, 20 ml of the EC degradation agent 2 and 20 ml of the EC degradation agent 3 prepared in Example 2 was used to treat a Baijiu sample containing EC, specifically as follows:
each of 20 ml of the EC degradation agent 1, 20 mL of the EC degradation agent 2 and 20 mL of the EC degradation agent 3 was added into 60 ml of Baijiu with 45% vol, and each of the mixtures was put into a 250 ml fermentation flask to react at 28° C. for 5 d under the condition of 100 rpm.

Control group: 20 ml of a YPD medium was added into 60 mL of Baijiu with 45% vol, and the mixture was put into a 250 ml fermentation flask to react at 28° C. for 5 d under the condition of 100 rpm.

After the reactions were finished, 2 mL of supernatant obtained from each of the reactions was sucked out and filtered with a 0.2 μm filter membrane, and the residual amount of EC in the culture supernatant was measured, with 3 replicates for each treatment. The results are shown in Table 1.

TABLE 1
EC degrading rates of different EC degradation agents

| Sample name | EC concentration (μg/L) | Degrading rate (%) |
|---|---|---|
| Control group | 230.3737 | \ |
| EC degradation agent 1 | 86.9693 | 62.24 |
| EC degradation agent 2 | 100.0902 | 56.56 |
| EC degradation agent 3 | 55.3112 | 76.00 |

The results show that the EC degradation agent 1, the EC degradation agent 2 and the EC degradation agent 3 can all reduce the EC concentration in Baijiu from 230.3737 μg/L to 86.9693 μg/L, 100.0902 μg/L and 55.3112 μg/L respectively, with the degrading rates being 62.24%, 56.56% and 76.00%.

It can be seen that the EC degradation agent 3 has the best degrading effect on EC in Baijiu.

### Example 4: Experiment of EC Degradation Agent 3 Prepared from R. toruloides DL-XSY01 in Degradation of EC Added to Fermented Food

In the following examples, EC degrading agent 3 was taking as an example to prove that the strain of this application can degrade EC in various fermented foods and alcoholic beverages. The samples used below are all commercially available products.

The treatment method of the EC degradation agent 3 involved in the following example included: a strain R. toruloides DL-XSY01 was inoculated into a YPD liquid medium for activation, and constant temperature shake cultivation was carried out at 25-30° C. for 24 h at a speed of 150-250 rpm to obtain seed fermentation broth; the seed fermentation broth was inoculated into a production fermentation tank (70% liquid filling capacity) containing a YPD fermentation medium at an inoculation amount of 10%, with a ventilation rate being 0.6-1.0 m³/min, and a stirring rate being 300 rpm, the cultivation temperature was controlled at 28° C., and cultivation lasted for 48-96 h; after fermentation was finished, a DL-XSY01 culture solution with a total bacterial count of 3×10⁸-1.8×10⁹ cfu/mL was obtained; after the fermentation was completed, the DL-XSY01 culture solution collected was centrifuged at a speed of 4000 g for 10 min to collect DL-XSY01 bacterial cell sediment; the bacterial cell sediment was diluted with a diluent PBS buffer solution, so as to obtain strain DL-XSY01 bacterial cell suspension, where the bacterial concentration was OD₆₀₀=20; the bacterial cell suspension was subjected to cell wall breaking to obtain DL-XSY01 cell lysate; then, sodium alginate (4%) was mixed with the DL-XSY01 cell lysate at a volume ratio of 1:1; the mixture was sucked with a 10 ml syringe and slowly dripped into a curing solution (a saturated boric acid solution containing 0.6% CaCl₂)) at a rate of 2-10 drops per second, with a curing time being about 5 h, so as to obtain immobilized cells; the immobilized cells were rinsed for 3-4 times with normal saline, and then placed in a chitosan (2%) solution for 40 min of film coating; and the product was rinsed again with 0.8% normal saline for 3-4 times, the water was drained off, and the obtained product was stored at 4° C. for later use.
1. Preparation of EC reaction solution for red wine: 60 ml of red wine (12% vol), 3.0 ppm EC, and 20 ml of an EC degradation agent
3 were incubated in a 250 ml conical flask at 28° C. for 5 d under the condition of 100 rpm.
2. Preparation of EC reaction solution for yogurt:
60 mL of yogurt, 3.0 ppm EC, and 20 ml of an EC degradation agent 3 were incubated in a 250 ml conical flask at 28° C. for 5 d under the condition of 100 rpm.
3. Preparation of EC reaction solution for vinegar:
60 ml of vinegar, 3.0 ppm EC, and 20 ml of an EC degradation agent 3 were incubated in a 250 ml conical flask at 28° C. for 5 d under the condition of 100 rpm.
4. Preparation of EC reaction solution for soy sauce:
60 ml of soy sauce, 3.0 ppm EC, and 20 ml of an EC degradation agent 3 were incubated in a 250 ml conical flask at 28° C. for 5 d under the condition of 100 rpm.
5. Preparation of EC reaction solution for sauce:
30 g of sauce added with 30 mL of deionized water, 3.0 ppm EC, and 20 ml of an EC degradation agent 3 were incubated in a 250 ml conical flask at 28° C. for 5 d under the condition of 100 rpm.

2 mL of supernatant of each of the above prepared EC reaction solutions for the fermented foods was sucked out after the reactions, and then filtered with a 0.2 μm filter membrane to measure the residual amount of EC in the culture supernatant. At the same time, an inoculation-free YPD medium was set as a control, with 3 replicates for each treatment. The results are shown in Table 2.

TABLE 2
EC concentrations after 5 d of reactions

| Content | Red wine (ethanol | Non-curdled | | Soy | |
|---|---|---|---|---|---|
| (ppm) | (12% vol)) | yogurt | Vinegar | sauce | Sauce |
| Blank group | 3.0 | 3.0 | 3.0 | 3.0 | 3.0 |
| Treatment | 2.3 | 2.4 | 2.2 | 2.2 | 2.6 |
| for 24 h | | | | | |
| Treatment | 1.6 | 1.8 | 1.6 | 1.3 | 1.8 |
| for 72 h | | | | | |
| Treatment | 0.7 | 0.9 | 0.6 | 0.6 | 1.0 |
| for 120 h | | | | | |
| Final | 76.67% | 70.00% | 80% | 80% | 66.67% |
| degrading | | | | | |
| rate | | | | | |

The results show that the DL-XSY01 fermented products and their EC degradation agents have a good degrading effect on EC, while the ethanol content, salt and other components in the fermented foods have little effect on the EC degradation activity of the DL-XSY01.

### Example 5: Application of R. toruloides DL-XSY01

In the following example, an EC degradation agent 3 was taken as an example to prove that the strain provided by the present application could degrade EC in actual alcoholic beverages. The samples used below are all commercially available products.

The treatment method of the EC degradation agent 3 involved in the following example was the same as that in Example 4.
1. Preparation of EC reaction solution for sesame-flavor Baijiu:
60 mL of sesame-flavor Baijiu (53% vol) and 20 ml of an EC degradation agent 3 were incubated in a 250 ml conical flask at 28° C. for 2 d under the condition of 100 rpm.
2. Preparation of EC reaction solution for Luzhou-flavor Baijiu:
60 mL of Luzhou-flavor Baijiu (45% vol) and 20 mL of an EC degradation agent 3 were incubated in a 250 ml conical flask at 28° C. for 2 d under the condition of 100 rpm.
3. Preparation of EC reaction solution for Maotai-flavor Baijiu:
60 mL of Maotai-flavor Baijiu (42% vol) and 20 ml of an EC degradation agent 3 were incubated in a 250 ml conical flask at 28° C. for 2 d under the condition of 100 rpm.
4. Preparation of EC reaction solution for Fen-flavor Baijiu:
60 mL of Fen-flavor Baijiu (38% vol) and 20 mL of an EC degradation agent 3 were incubated in a 250 ml conical flask at 28° C. for 2 d under the condition of 100 rpm.
5. Preparation of EC reaction solution for Huangjiu:
60 mL of Huangjiu (15% vol) and 20 ml of an EC degradation agent 3 were incubated in a 250 ml conical flask at 28° C. for 2 d under the condition of 100 rpm.

2 mL of supernatant of each of the above prepared EC reaction solutions for the fermented foods was sucked out after the reactions, and then filtered with a 0.2 μm filter membrane to measure the residual amount of EC in the culture supernatant. At the same time, an inoculation-free YPD medium was set as a control, with 3 replicates for each treatment. The results are shown in Table 3.

TABLE 3
EC concentrations in actual alcoholic
beverages after 2 d of reactions

| | Sesame- | Luzhou- | Maotai- | Fen- | |
|---|---|---|---|---|---|
| | flavor | flavor | flavor | flavor | |
| EC content | Baijiu | Baijiu | Baijiu | Baijiu | Huangjiu |
| (μg/L) | (53% vol) | (45% vol) | (42% vol) | (38% vol) | (15% vol) |
| Initial | 214.1 | 191.9 | 72.8 | 46.2 | 307.2 |
| concentra- | | | | | |
| tion | | | | | |
| After 48 h of | 53.2 | 45.3 | 23.1 | 20.1 | 89.9 |
| treatment | | | | | |
| Degrading | 75.15% | 76.39% | 63.65% | 56.56% | 70.74% |
| rate | | | | | |

The results show that the DL-XSY01 fermented products and their EC degradation agents have a good degrading effect on EC in actual fermentation samples, and the degrading rates of EC in alcoholic beverages can reach about 70%.

### Example 6: Application of R. toruloides DL-XSY01 in Reduction of EC Content During Fermentation

In the following example, DL-XSY01 was used to treat vinegar fermented grains during fermentation, which proved that the strain provided by the present application can reduce the production of EC during fermentation.

A preparation method of the DL-XSY01 involved in the following example was as follows: a strain R. toruloides DL-XSY01 was inoculated into a YPD liquid medium for activation, and constant temperature shake cultivation was carried out at 25-30° C. for 24 h at a speed of 150-250 rpm to obtain seed fermentation broth; the seed fermentation broth was inoculated into a production fermentation tank (70% liquid filling capacity) containing a YPD fermentation medium at an inoculation amount of 10%, with a ventilation rate being 0.6-1.0 m³/min, and a stirring rate being 300 rpm, the cultivation temperature was controlled at 28° C., and cultivation lasted for 48-96 h; after fermentation was finished, a DL-XSY01 culture solution with a total bacterial count of 3×10⁸-1.8×10⁹ cfu/mL was obtained; and after fermentation was completed, the DL-XSY01 culture solution collected was subjected to freeze vacuum drying to obtain dry bacterial cells.

5 g of the freeze-dried bacterial cells were taken and added into 100 g of vinegar fermented grains for 20 d of fermentation so as to obtain white vinegar that had been inoculated and fermented. In addition, 100 g of vinegar fermented grains were directly fermented for 20 d to obtain uninoculated white vinegar.

2 mL of each of the white vinegar that had been inoculated and fermented and the uninoculated white vinegar was sucked out to obtain a maximum EC residual amount, with 3 replicates for each group of samples. The results are shown in Table 4.

TABLE 4
EC content in white vinegar before and
after inoculation and fermentation

| Sample name | EC concentration (μg/L) |
|---|---|
| White vinegar subjected to | 21.3 |
| inoculation and fermentation | |
| White vinegar not subjected to | 31.5 |
| inoculation and fermentation | |

The results show that after the addition of the freeze-dried DL-XSY01 bacterial cells, the concentration of EC in a fermentation process can be reduced.

### Example 7: Properties of R. toruloides DL-XSY01 Strain

The Specific Steps were as Follows:

1. Safety evaluation of DL-XSY01 strain

### (1) Antibiotic Sensitivity Test

The antibiotic sensitivity spectrum of the R. toruloides DL-XSY01 was characterized by using a disc diffusion method.

Single colonies of the DL-XSY01 were selected from a YPD-agar solid plate and transferred to a YPD liquid medium for culture, so as to obtain a bacterial solution (about 1×10⁸ cfu/mL);

200 μL of the bacterial solution of the DL-XSY01 was taken and spread onto a YPD-agar solid plate; 11 types of antibiotic sensitivity paper containing gentamicin (10 μg), streptomycin (10 μg), erythromycin (15 μg), tetracycline (30 μg), cephalexin (30 μg), vancomycin (30 μg), cefazolin (30 μg), ampicillin (10 μg), penicillin (10 μg), minocycline (30 μg) and amikacin (30 μg) were slowly placed onto the solid plate, respectively; the distance between every two adjacent pieces of paper was not less than 24 mm;

The above medium with each type of paper was statically cultured at 28° C. for 24 h, and then the diameter of an inhibition zone was measured and counted; and analysis was performed on the drug resistance R (≤14 mm), intermediate-phase I (14-20 mm) or sensitivity S (≥20 mm), with three replicates for each group. The antibiotic sensitivity of the DL-XSY01 is shown in Table 5.

TABLE 5
Antibiotic sensitivity of DL-XSY01

| Antibiotic | DL-XSY01 |
|---|---|
| Cefazolin | S |
| Erythromycin | S |
| Gentamicin | S |
| Tetracycline | S |
| Amikacin | S |
| Ampicillin | S |
| Streptomycin | S |
| Vancomycin | S |
| Minocycline | S |
| Cephalexin | I |
| Penicillin G | S |

The results show that the strain is moderately sensitive to the antibiotic cephalexin, and sensitive to 10 antibiotics including cefazolin, erythromycin, gentamicin, tetracycline, amikacin, ampicillin, streptomycin, vancomycin, minocycline, and penicillin G. Therefore, according to the CLSI guidelines, these results confirm that the DL-XSY01 is safe.

### (2) Hemolysis Experiment

Hemolysis may be divided into three types, of which a hemolysis is also called grass green hemolysis. The appearance of a 1-2 mm grass green ring in a medium around colonies is caused by methemoglobin, and red blood cells in the a hemolysis ring are not completely dissolved, which may form bacteria, such as α-hemolytic streptococcus and Streptococcus pneumoniae, in the a hemolysis ring. B hemolysis is the formation of a wide (2-4 mm), clearly defined, and completely transparent hemolysis ring around colonies during culturing on a solid plate and the complete dissolution of red blood cells in the ß hemolysis ring; and the red blood cells are completely dissolved by hemolysin produced by bacteria, also known as complete hemolysis. The bacteria capable of forming the ß hemolytic ring include β-hemolytic streptococcus, S. aureus, and the like. γ hemolysis is also known as non-hemolysis, there is no hemolysis ring around the colonies.

The specific steps were as follows:

Preparation of medium: Columbia agar+5% defibrated sheep blood.

Single colonies of the DL-XSY01 were selected from a YPD-agar solid plate and transferred to a YPD liquid medium for culture, so as to obtain a bacterial solution (about 1×10⁸ cfu/mL).

The bacterial solution was streaked in a Columbia plate containing 5% defibrated sheep blood, and cultured at 28° C. for 24 h; and then, whether there was a transparent circle was observed. S. aureus ATCC 25923 was used as a positive control, with three replicates for each group.

The results showed that S. aureus ATCC 25923 was used as a positive control in this hemolysis experiment. DL-XSY01 do not show hemolysis (γ-hemolysis) when being cultured on the sheep blood plate at 28° C. However, S. aureus ATCC 25923 showed β-hemolysis, and thus the positive control was established. In addition, E. coli Nissle 1917 showed α-hemolysis. Therefore, DL-XSY01 is considered a safe organism that poses no harm to human health.

### 2. Probiotic Evaluation of DL-XSY01 Strain

(1) Artificially simulated gastric juice experiment

In order to colonize the gastrointestinal tract and play a probiotic role, probiotics must first have a certain tolerance to the digestive tract environment, and the tolerance to gastric juice is an important screening criterion.

Preparation of simulated gastric juice: 16.4 mL of diluted hydrochloric acid added with about 900 ml of water, and 10 g of pepsin were mixed together evenly, and then water was added into the mixture to make up to 1000 mL. The pH was adjusted to 2.5, and the product was sterilized by filtration with a 0.22 μm filter membrane and then stored at 4° C.

### Simulated Gastric Juice Tolerance Experiment:

Single colonies of the DL-XSY01 were selected from a YPD-agar solid plate and transferred to a YPD liquid medium for culture, so as to obtain a bacterial solution (about 1×10⁸ cfu/mL). 5 mL of the bacterial solution was taken and centrifuged at 10000 rpm for 5 min at 4° C. to collect bacterial cells, the bacterial cells were washed twice with a sterile phosphate buffer at pH of 7.4, and the above operation was repeated.

The bacterial cells were resuspended in 5 mL of artificially simulated gastric juice at pH of 2.5. A spread plate method was used to calculate the number of viable bacteria incubated in the artificially simulated gastric juice at pH of 2.5 for 0 h and 3 h, with three replicates for each group.

Survival rate (%)=log CFU(N1)/log CFU(N0)*100%.

In the formula, N0 represents the number of viable bacteria incubated in artificially simulated gastric juice for 0 h, and N1 represents the number of viable bacteria incubated in artificially simulated gastric juice for 3 h.

Meanwhile, E. coli Nissle 1917 was used as a positive control.

The results showed that the survival rate of DL-XSY01 was 86.73% and the survival rate of E. coli Nissle 1917 in the positive control group was 86.14% after cultured for 3 h under artificially simulated gastric juice conditions (containing 0.3% pepsin, with pH value being 2.5). The results indicate that compared with E. coli Nissle 1917, DL-XSY01 has a higher survival rate in artificially simulated gastric juice.

### (2) Artificially Simulated Intestinal Juice Experiment

In order to colonize the gastrointestinal tract and play a probiotic role, probiotics must first have a certain tolerance to the digestive tract environment, and the tolerance to intestinal juice is an important criterion.

Preparation of simulated intestinal juice: trypsin was prepared into a solution with a concentration of 1 mg/ml by using a sterilized phosphate buffer (pH 7.4), 0.3% bile salt from ox was added, and the pH value was adjusted to 7.4 with 1 mol/L sodium hydroxide, and then the product was sterilized by filtration with a 0.22 μm microporous filter membrane for later use.

Simulated intestinal juice tolerance experiment:

Single colonies of the DL-XSY01 were selected from a YPD-agar solid plate and transferred to a YPD liquid medium for culture, so as to obtain a bacterial solution (about 1×10⁸ cfu/mL).

5 mL of the bacterial solution was taken and centrifuged at 10000 rpm for 5 min at 4° C. to collect bacterial cells, the bacterial cells were washed twice with a sterile phosphate buffer at pH of 7.4, and the above operation was repeated. The bacterial cells were resuspended in 5 mL of artificially simulated intestinal juice at pH of 7.4. A spread plate method was used to calculate the number of viable bacteria incubated in the artificially simulated intestinal juice at pH of 7.4 for 0 h and 4 h, with three replicates for each group.

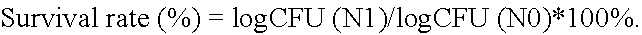

In the formula, N0 represents the number of viable bacteria incubated in artificially simulated intestinal juice for 0 h, and N1 represents the number of viable bacteria incubated in artificially simulated intestinal juice for 4 h.

Meanwhile, E. coli Nissle 1917 was used as a positive control.

The results showed that the survival rate of DL-XSY01 was 85.92% and the survival rate of E. coli Nissle 1917 in the positive control group was 84.64% after cultured for 4 h under artificially simulated intestinal juice conditions (containing 0.3% pepsin and 0.3% bile salt from ox). The results indicate that compared with E. coli Nissle 1917, DL-XSY01 has a higher survival rate in artificially simulated intestinal juice.

### (3) Antioxidant Experiment

DPPH free radical scavenging rate test:

0.0078 g of DPPH was dissolved in anhydrous ethanol, and the volume was made up to 100 mL. 0.2 mmol/L DPPH was prepared, which was kept away from light for immediate use.

The DL-XSY01 bacterial solution with a bacterial concentration of 10⁸ cfu/ml was prepared according to the above step (1).

The above 10⁸ cfu/mL DL-XSY01 bacterial solution was mixed with 100% ethanol DPPH solution (0.2 mM) at a volume ratio of 1:1, and the mixture was incubated in the dark at 25° C. for 30 min.

The DL-XSY01 bacterial solution and 100% ethanol were used alone as a blank, and the DPPH ethanol solution was used as a control. The supernatant was collected after centrifugation at 2330×g (4120 rpm) for 10 min. Absorbance was measured at 517 nm in triplicate.

ABTS free radical scavenging rate test:

ABTS (14 mM) and potassium persulfate (5 mM) were dissolved in 0.1 M potassium phosphate buffer (pH 7.4), mixed at a ratio of 1:1, and reacted at 25° C. for 12-16 h.

100 μl of a strain DL-BJ01 (10⁸ cfu/mL) was added to 900 μL of ABTS solution, and incubated at 25° C. in the dark for 15 min. After centrifugation (at 14000 g for 1 min), the absorbance of the supernatant was measured at 734 nm.

The results showed that the DPPH scavenging activity of DL-XSY01 was 90.02%, and the ABTS scavenging activity thereof was 93.67%, which indicated that the strain DL-XSY01 had good antioxidant activity.

Although the present disclosure has been disclosed as above in exemplary examples, it is not intended to limit the present disclosure. Anyone familiar with the art can make various changes and modifications without departing from the spirit and scope of the present disclosure. Therefore, the protection scope of the present disclosure shall be as defined in the claims.

What is claimed is:

1. A method of use of Rhodosporidium toruloides DL-XSY01 or its fermentation broth in the preparation of a product capable of degrading ethyl carbamate, comprising adding R. toruloides DL-XSY01 or its fermentation broth during preparation of the product capable of degrading ethyl carbamate, and the R. toruloides DL-XSY01 has been deposited in the China General Microbiological Culture Collection Center (CGMCC) on Oct. 8, 2021, with a preservation number of CGMCC No. 23534.

2. The method according to claim 1, wherein the product is a chemical or a microbial agent.

3. The method according to claim 2, wherein the addition amount of the R. toruloides DL-XSY01 in the product is at least 3×10⁸ cfu/mL.

4. The method according to claim 3, wherein the chemical is an ethyl carbamate degradation agent.

5. The method according to claim 1, wherein the product is a food, further comprising sterilizing after addition of the R. toruloides DL-XSY01 or its fermentation broth to degrade ethyl carbamate to obtain a sterilized food.

6. The method according to claim 5, wherein the food is a fermented food or an alcoholic beverage.

7. The method according to claim 6, wherein the food is Baijiu, Huangjiu, red wine, yogurt, vinegar, or soy sauce.

8. An ethyl carbamate degradation agent, wherein the degradation agent is prepared according to the following method:
(1) inoculating the R. toruloides DL-XSY01 into a YPD liquid medium for activation, and carrying out constant temperature shake cultivation at 25-32° C. for 24-48 h at a speed of 150-250 rpm to obtain seed fermentation broth, and the R. toruloides DL-XSY01 has been deposited in the China General Microbiological Culture Collection Center (CGMCC) on Oct. 8, 2021, with a preservation number of CGMCC No. 23534;
(2) inoculating the seed fermentation broth into a fermentation medium at an inoculation amount of 10%-20%, and fermenting until the total bacterial count is 3×10⁸-1.8×10⁹ cfu/mL under the conditions that the temperature is 25-32° C., the ventilation rate is 0.6-1.0 m³/min and the stirring rate is 150-250 rpm, so as to obtain a fermentation product of the R. toruloides DL-XSY01; and
(3) embedding the fermentation product of the R. toruloides DL-XSY01 prepared in step (2) to obtain the ethyl carbamate degradation agent.
